(19) United States
(12) Patent Application Publication
(10) Pub. No.: US 2026/0124007 A1
(43) Pub. Date: May 7, 2026

(54) TROCAR HOLDER OF SURGICAL ROBOT

(71) Applicant: LIVSMED INC., Seongnam-si (KR)

(72) Inventor: Young Jae SONG, Seongnam-si (KR)

(21) Appl. No.: 19/377,241

(22) Filed: Nov. 3, 2025

(30) Foreign Application Priority Data
Nov. 5, 2024 (KR) 10-2024-0154960

(51) Int. Cl. A61B 34/30; A61B 17/34; A61B 34/00

(52) U.S. Cl. CPC A61B 34/30; A61B 17/34; A61B 34/70; A61B 2034/305

(57) ABSTRACT

A trocar holder of the surgical robot includes a grip assembly including a first grip portion and a second grip portion that are disposed to face each other and configured to move closer to or away from each other, a holder body to which at least a part of the grip assembly is connected, a power generation device including a driving motor configured to generate power for driving the grip assembly, and a power transfer device disposed in the holder body and configured to transfer the power generated by the driving motor to the grip assembly.

### CROSS-REFERENCE TO RELATED APPLICATION

This application is based on and claims priority under 35 U.S.C. § 119 to Korean Patent Application No. 10-2024-0154960, filed on Nov. 5, 2024, in the Korean Intellectual Property Office, the disclosure of which is incorporated by reference herein in its entirety.

### BACKGROUND

### 1. Field

The present disclosure relates to a trocar holder of a surgical robot.

### 2. Description of the Related Art

Surgery denotes a process of curing illness by cutting, incising, or manipulating the skin, the mucosa layer, and other tissues by using a medical instrument. In particular, laparotomy that treats, shapes, or removes an organ by cutting and opening the skin of a surgical site may cause bleeding, side effects, pain of a patient, scar, etc. Therefore, surgery performed by inserting only a medical instrument, e.g., a laparoscope, a surgical instrument, a microscope for microsurgery, etc. after forming a predetermined hole in the skin, or surgery using a robot has been recently considered as an alternative.

Here, a surgical robot refers to a robot capable of performing surgical action on behalf of a surgeon who has performed the surgical action. Such a surgical robot may perform accurate and precise operations as compared with human beings and enable a remote surgery.

Surgical robots that are currently being developed worldwide may include bone surgery robots, laparoscopic surgery robots, stereotactic surgery robots, etc. Here, a laparoscopic surgical robot denotes a robot performing a minimal invasive surgery using a laparoscope and surgical tools.

In addition, a surgical robot generally includes a master robot and a slave robot. When an operator manipulates a manipulation lever (e.g., a handle) provided on the master robot, an instrument that is coupled to a robot arm of the slave robot or held by the robot arm is manipulated to perform surgery.

Laparoscopic surgery is a cutting-edge surgical technique in which a laparoscope that is an endoscope for looking inside the abdomen after making a small hole in a navel area and then surgery is performed, and is expected to have a large development in the future. Recently, a laparoscope has been provided with a computer chip to obtain more clear and magnified images as compared with the images seen with naked eyes, and has developed so that, when specifically designed laparoscopic surgical instruments are used while watching a screen of a monitor, any kind of surgery may be performed.

In addition, the laparoscopic surgery is executed while observing a surgical site in the abdominal cavity by forming a tube connecting into the abdomen area of a patient by using a surgical tool called a trocar and making the surgical tool such as a laparoscope, a surgical instrument, etc. enter the surgical site in the abdominal cavity through the trocar.

The above-mentioned background technology is technical information that the inventor possessed for deriving the present disclosure or acquired in the process of deriving the present invention, and cannot necessarily be said to be known art disclosed to the general public before filing the application for the present disclosure.

### SUMMARY

The present disclosure provides a trocar holder of a surgical robot, the trocar holder being applied to a surgical robot to be used in laparoscopic surgery or other various surgery, which is capable of facilitating easy fastening of a trocar through an automated trocar fastening method and reducing an error that may occur during manipulation.

According to an embodiment of the present disclosure, a trocar holder of a surgical robot may include a grip assembly including a first grip portion and a second grip portion that are arranged to face each other and configured to move closer to or away from each other, a holder body to which at least a part of the grip assembly is connected, a power generation device including a driving motor for generating power for driving the grip assembly, and a power transfer device that is disposed in the holder body and configured to transfer the power generated by the driving motor to the grip assembly.

In another embodiment of the present disclosure, when a driving shaft of the driving motor is rotated in a first direction, the first grip portion and the second grip portion may be rotated in a direction approaching each other, and when the driving shaft is rotated in a second direction opposite to the first direction, the first grip portion and the second grip portion may be rotated in a direction away from each other.

In the other embodiment of the present disclosure, the power transfer device may allow the first grip portion and the second grip portion to be rotated facing each other while being arranged symmetrically with each other.

In the other embodiment of the present disclosure, the first grip portion and the second grip portion may be rotated coaxially.

In the other embodiment of the present disclosure, the first grip portion may rotate about a first rotary shaft, and the second grip portion may rotate about a second rotary shaft that is arranged to be spaced apart from the first rotary shaft.

In the other embodiment of the present disclosure, the first rotary shaft may be axially coupled to a proximal end of the first grip portion and the holder body, and the second rotary shaft may be axially coupled to a proximal end of the second grip portion and the holder body.

In the other embodiment of the present disclosure, the trocar holder may further include a trocar sensor configured to sense a trocar attached to the trocar holder.

In the other embodiment of the present disclosure, the driving motor may be automatically operated by a signal provided by the trocar sensor when the trocar sensor senses the trocar.

In the other embodiment of the present disclosure, the trocar sensor may be arranged between a first rotary shaft of the first grip portion and a second rotary shaft of the second grip portion.

In the other embodiment of the present disclosure, the first grip portion may include a first point-of-force portion that extends from a first rotating center of the first grip portion and receives a force for rotating the first grip portion at a position spaced apart from the first rotating center of the first grip portion, and the second grip portion may include a second point-of-force portion that extends from a second rotating center of the second grip portion and receives a force for rotating the second grip portion at a position spaced apart from the second rotating center of the second grip portion.

In the other embodiment of the present disclosure, the power transfer device may include a first connection member connected to the first point-of-force portion and a second connection member connected to the second point-of-force portion.

In the other embodiment of the present disclosure, the first connection member and the second connection member may receive the power from the power generation device and apply force to the first point-of-force portion and the second point-of-force portion, respectively, so that the first grip portion and the second grip portion may externally rotate or internally rotate.

In the other embodiment of the present disclosure, the first grip portion may include a third rotary shaft inserted through the first connection member and the first point-of-force portion to be a rotating center of the first connection member, and a fourth rotary shaft inserted through the second connection member and the second point-of-force portion to be a rotating center of the second connection member.

In an embodiment of the present disclosure, the first grip portion may rotate about the first rotary shaft and the second grip portion may rotate about the second rotary shaft, the third rotary shaft may be parallel to the first rotary shaft, and the fourth rotary shaft may be parallel to the second rotary shaft.

In the other embodiment of the present disclosure, the first point-of-force portion may include a first point-of-force upper end portion and a first point-of-force lower end portion that are protruding regions, and the first connection member may be at least partially accommodated between the first point-of-force upper end portion and the first point-of-force lower end portion.

In the other embodiment of the present disclosure, movable coupling holes may be formed in regions at the proximal end sides of the first grip portion and the second grip portion, and protrusions may be formed on a first plate of the holder body so that the protrusions may relatively move within the movable coupling holes while the protrusions are inserted into the movable coupling holes.

In the other embodiment of the present disclosure, the power transfer device may include one shaft connected to the power generation device and defining a power transfer axis, a linear movement portion configured to perform translational movement between a proximal end and a distal end along the power transfer axis, a first connection member connecting the linear movement portion to the first grip portion, and a second connection member connecting the linear movement portion to the second grip portion.

In the other embodiment of the present disclosure, the first grip portion and the second grip portion may be configured to perform an opening operation in which the first grip portion and the second grip portion are spaced apart from each other when the linear movement portion moves toward the proximal end side, and to perform a closing operation in which the grip portion and the second grip portion approach closer to each other when the linear movement portion moves toward the distal end side.

In the other embodiment of the present disclosure, the linear movement portion may perform translational movement when the one shaft rotates about the power transfer axis.

In the other embodiment of the present disclosure, the linear movement portion may include a nut portion through which the one shaft is inserted, and a first hinge portion and a second hinge portion provided at both sides of the nut portion, wherein one end portion of the first connection member may be rotatably coupled to the first hinge portion and another end portion of the first connection member is rotatably coupled to the first grip portion, and one end portion of the second connection member may be rotatably coupled to the second hinge portion and another end portion of the second connection member is rotatably coupled to the second grip portion.

In the other embodiment of the present disclosure, the power transfer device may include one shaft including a pinion gear, connected to the power generation device, and defining a power transfer axis, a first rack and a second rack that are arranged to face each other with the pinion gear interposed therebetween, where each of the first rack and the second rack including a gear engaged with the pinion gear, a first lever member connected to the first rack, a second lever member connected to the second rack, a first connection member connecting the first lever member to the first grip portion, and a second connection member connecting the second lever member to the second grip portion.

In the other embodiment of the present disclosure, the first lever member may have one end portion extending from a first rotating center of the first lever member and connected to the first rack, and another end portion extending from the first rotating center of the first lever member and hinge-coupled to the first connection member, and the second lever member may have one end portion extending from a second rotating center of the second lever member and connected to the second rack, and another end portion extending from the second rotating center of the second lever member and hinge-coupled to the second connection member.

In the other embodiment of the present disclosure, a first elongated hole may be defined in one end portion of the first lever member and a first protrusion may be disposed on one end portion of the first rack, so that the first protrusion may move a certain degree within the first elongated hole while the first protrusion is inserted in the first elongated hole, and a second elongated hole ma be defined in one end portion of the second lever member and a second protrusion may be disposed on one end portion of the second rack, so that the second protrusion may move a certain degree within the second elongated hole while the second protrusion is inserted in the second elongated hole.

In the other embodiment of the present disclosure, the first rack and the second rack may be arranged to intersect a power transfer axis of the one shaft, and upon rotation of the one shaft in one direction, the first rack may move in a longitudinal direction of the first rack, and the second rack may move in a longitudinal direction of the second rack, so that one end portion of the first rack and one end portion of the second rack may move closer to or move away from each other.

In the other embodiment of the present disclosure, when one end portion of the first rack, which is connected to the first lever member, and one end portion of the second rack, which is connected to the second lever member, approach closer to each other due to the rotation of the one shaft in one direction, an opening operation in which the first grip portion and the second grip portion are spaced apart from each other may be performed.

In the other embodiment of the present disclosure, when the first lever member is rotated so that one end portion at the proximal end side of the first lever member approaches the one shaft and the other end portion of the first lever member is away from the one shaft, the first connection member connected to the other end of the first lever member may externally rotate the first grip portion.

Other aspects, features and advantages other than those described above will become apparent from the following detailed description of the drawings, claims and disclosure.

### BRIEF DESCRIPTION OF THE DRAWINGS

These and/or other aspects will become apparent and more readily appreciated from the following description of the embodiments, taken in conjunction with the accompanying drawings.

FIG. 1 is a conceptional diagram showing a surgical robot system provided with a surgical robot according to an embodiment of the present disclosure.

FIG. 2 is a perspective view of the surgical robot of FIG. 1.

FIG. 3 is a perspective view showing a magnified view of some components of a first arm unit of FIG. 2.

FIG. 4 is a schematic diagram of a trocar holder according to an embodiment of the present disclosure.

FIG. 5 is a schematic diagram of a trocar holder according to another embodiment of the present disclosure.

FIG. 6 is a perspective view of a trocar holder and a trocar attached to the trocar holder according to an embodiment of the present disclosure.

FIG. 7 is a perspective view showing a magnified view of the trocar holder of FIG. 6.

FIG. 8 is a perspective view showing a state in which a first plate is removed from the trocar holder of FIG. 7.

FIG. 9 is a diagram of a grip assembly and the first plate of FIG. 7.

FIG. 10 is a plan view of the trocar holder of FIG. 8.

FIG. 11 is a plan view for illustrating an opening operation of the trocar holder of FIG. 10.

FIG. 12 is a perspective view of a trocar holder according to another embodiment of the present disclosure.

FIG. 13 is a perspective view showing a state in which an upper plate is removed from the trocar holder of FIG. 12.

FIG. 14 is a perspective view showing some components of the trocar holder of FIG. 13 from a different angle.

FIG. 15 is a plan view of the trocar holder of FIG. 13.

FIG. 16 is a plan view for illustrating an opening operation of the trocar holder of FIG. 15.

### DETAILED DESCRIPTION

The embodiments will be described below in more detail with reference to the accompanying drawings. Those components that are the same or are in correspondence are rendered the same reference numeral regardless of the figure number, and redundant explanations are omitted.

As the present disclosure allows for various changes and numerous embodiments, particular embodiments will be illustrated in the drawings and described in detail in the written description. The attached drawings for illustrating one or more embodiments are referred to in order to gain a sufficient understanding, the merits thereof, and the objectives accomplished by the implementation. However, the embodiments may have different forms and should not be construed as being limited to the descriptions set forth herein.

In the description, certain detailed explanations of the related art are omitted when it is deemed that they may unnecessarily obscure the essence of the present disclosure.

An expression used in the singular encompasses the expression of the plural, unless it has a clearly different meaning in the context. It will be understood that although the terms “first,” “second,” etc. may be used herein to describe various components, these components should not be limited by these terms. These components are only used to distinguish one component from another. These components are only used to distinguish one component from another.

In the present specification, it is to be understood that the terms such as “including,” “having,” and “comprising” are intended to indicate the existence of the features or components disclosed in the specification, and are not intended to preclude the possibility that one or more other features or components may exist or may be added.

It will be understood that when a unit, region, or component is referred to as being “formed on” another layer, region, or component, it can be directly or indirectly formed on the other layer, region, or component. That is, for example, intervening units, regions, or components may be present.

In the embodiment below, the terms such as “below”, “lower”, “above”, “upper”, etc. are used to describe the relationships between components shown in the drawings. The above terms are relative concepts and described based on the directions shown in the drawings.

It will be understood that when an element is referred to as being “connected” or “coupled” to another element, it can be directly connected or coupled to the other element or intervening elements may be present.

Sizes of components in the drawings may be exaggerated for convenience of explanation. In other words, since sizes and thicknesses of components in the drawings are arbitrarily illustrated for convenience of explanation, the following embodiments are not limited thereto.

A surgical robot 10 that may adopt a trocar holder according to an embodiment of the present disclosure is described below.

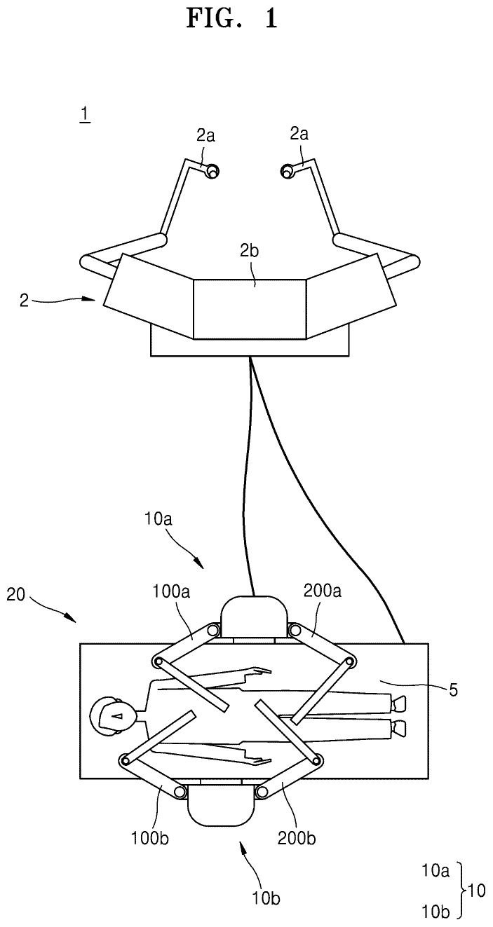

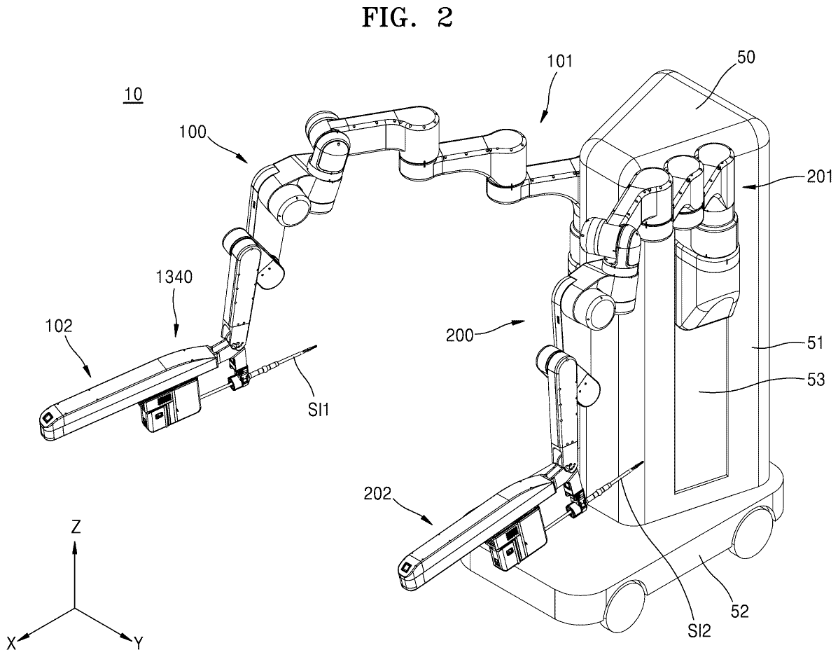

FIG. 1 is a conceptual diagram showing a surgical robot system provided with a surgical robot according to an embodiment of the present disclosure. FIG. 2 is a perspective view of the surgical robot of FIG. 1.

Referring to FIG. 1 and FIG. 2, the surgical robot system 1 may include a master robot 2 and the surgical robot 10.

The master robot 2 includes a manipulation member and a display member, and the surgical robot 10 includes one or more robot arm units 100 and 200.

In detail, the master robot 2 includes manipulation members 2a that may be held and manipulated by both hands of an operator. In addition, images captured by a laparoscope are displayed on a display member 2b of the master robot 2. Also, the display member 2b may display a certain virtual manipulation plate along with the images captured by the laparoscope, etc. or may independently display the virtual manipulation plate. As described above, detailed descriptions about the arrangement, structure, etc. of the virtual manipulation plate are omitted.

In addition, the surgical robot 10 may include at least two robot arm units 100 and 200. Here, each of the robot arm units 100 and 200 may be each provided as a module type that may independently operate, and an algorithm for preventing collision between the robot arm units 100 and 200 may be applied to the surgical robot system 1.

The surgical robot system 1 may include one or more surgical robots 10. FIG. 1 shows an example in which the surgical robot system 1 includes two surgical robots 10a and 10b, and each of the surgical robots 10a and 10b includes two robot arm units 100 and 200. Thus, total of four robot arm units 100a, 200a, 100b, and 200b are arranged.

In an embodiment, surgical instruments SI may be attached to two or more of the robot arm units 100 and 200, and a laparoscope may be attached to one or more of the robot arm units 100 and 200. In addition, a surgeon may select one or more of the robot arm units 100a, 200a, 100b, and 200b that is to be controlled via the master robot 2. As described above, the surgeon may directly manipulate a total of three or more surgical instruments via the master robot 2, and thus, various tools may be precisely and freely manipulated according to the intention of the surgeon on an operation bed 5 without any surgery assistant.

Hereinafter, detailed configurations and operating principles of the surgical robot 10 are described in detail below.

Referring to FIG. 2, the surgical robot 10 may include a main body 50, the first arm unit 100, and the second arm unit 200. FIG. 2 shows an example in which the surgical robot 10 includes two robot arm units, and hereinafter, respective robot arm units are defined as the first arm unit 100 and the second arm unit 200.

The main body 50 acts as a body of the surgical robot 10, and on the main body 50, the first arm unit 100 and the second arm unit 200 may be arranged. Also, the main body 50 may provide a reference point of driving the first arm unit 100 and the second arm unit 200.

The main body 50 may include a first main body 51 and a second main body 52. The first arm unit 100 and the second arm unit 200 are arranged on the first main body 51, and the second main body 52 may support the first main body 51. Also, the second main body 52 may have wheels as shown in FIG. 2, and accordingly, the surgical robot 10 may be moved by means of the wheels.

The main body 50 may have vertical movement guides 53. The number of vertical movement guides 53 may correspond to the number of robot arm units arranged on the main body 50. The vertical movement guide 53 may be formed concavely in one side of the main body 50, and respective robot arm units 100 and 200 may be coupled to the vertical movement guides 53 to slidably move in a first direction.

When describing the present disclosure, a part close to the main body 50 is referred to as a proximal end and a part far from the main body 50 is referred to as a distal end. For example, in the first arm unit 100, a part close to the main body 50 is defined as a proximal end 101 of the first arm unit 100, and a part far from the main body 50 is defined as a distal end 102 of the first arm unit 100. Likewise, in the second arm unit 200, a part close to the main body 50 is defined as a proximal end 201 of the second arm unit 200, and a part far from the main body 50 is defined as a distal end 202 of the second arm unit 200.

The first arm unit 100 is arranged on one side of the main body 50 and a first surgical instrument SI1 may be attached to the first arm unit 100. The surgical robot 10 drives the first arm unit 100 to adjust a position and posture of the first surgical instrument SI1.

A first arm connection portion (not shown) may include a plurality of connection links, and posture of the first arm connection portion may be determined according to the driving of each connection link. A remote center of motion (RCM) point RCM1 of the first surgical instrument SI1 may be determined according to the posture of the first arm connection portion. Here, the RCM point of the first surgical instrument SI1 denotes a virtual center point that becomes a base of the rotation of the first surgical instrument SI1. The first surgical instrument SI1 may rotate about the RCM point to perform a yaw movement and a pitch movement.

The second arm unit 200 is arranged on the other side of the main body 50 and a second surgical instrument SI2 may be attached on the second arm unit 200. The surgical robot 10 drives the second arm unit 200 to adjust a position and posture of the second surgical instrument SI2.

A second arm connection portion (not shown) may include a plurality of connection links, and posture of the second arm connection portion may be determined according to the driving of each connection link. A remote center of motion (RCM) point (not shown) of the second surgical instrument SI2 may be determined according to the posture of the second arm connection portion. Here, the RCM point of the second surgical instrument SI2 denotes a virtual center point that becomes a base of the rotation of the second surgical instrument SI2. The second surgical instrument SI2 may rotate about the RCM point to perform a yaw movement and a pitch movement.

Each of the arm units may include a plurality of arm connection links and a plurality of arm extension links. The arm connection link and the arm extension link may each rotate about a reference axis, and due to the rotation operation, the arm unit may adjust a posture and arrangement of the surgical instrument within a movable range.

Hereinafter, detailed descriptions about the arm connection links and the arm extension links are omitted.

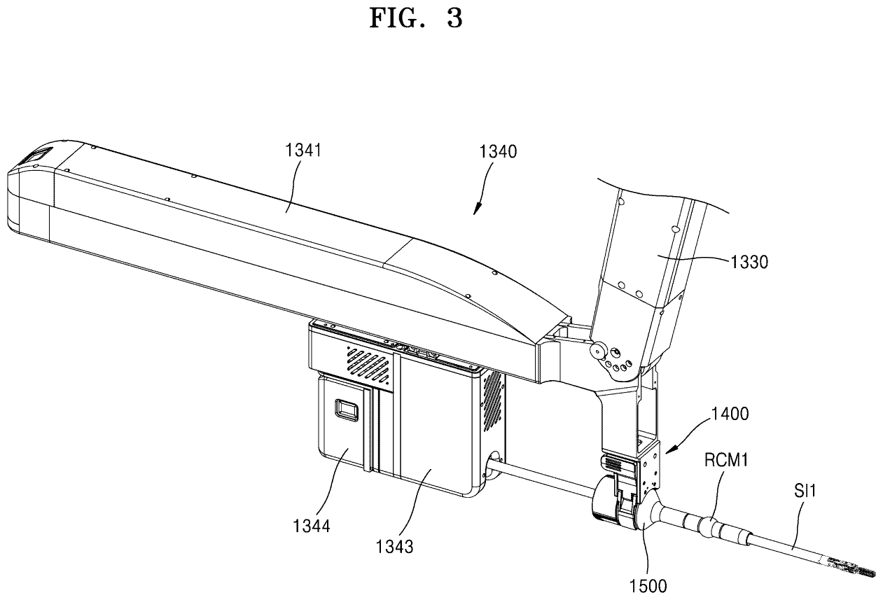

FIG. 3 is a perspective view showing a magnified view of some components of the first arm unit 100 of FIG. 2.

Referring to FIG. 2 and FIG. 3, the first arm unit 100 according to an embodiment may include a first arm slide link 1340 at a distal end 102 side.

The first arm slide link 1340 may allow the first surgical instrument SI1 to slidably move.

The first arm slide link 1340 may be coupled to the other end of a first arm third extension link 1330, that is, the distal end side, and the first surgical instrument SI1 may be arranged on the first arm slide link 1340.

The first arm slide link 1340 may include a translation arm 1341, a slide motor pack 1343, a slide driving portion 1344, and a trocar holder 1400.

The translation arm 1341 is coupled to the other end of the first arm third extension link 1330 and may be moved along with the first arm third extension link 1330. That is, when the first arm third extension link 1330 is driven, the posture of the translation arm 1341 may be also changed.

The slide motor pack 1343 may provide a driving force for sliding movement of the first surgical instrument SI1. The slide motor pack 1343 may include various components that may generate and transfer driving power, including one or more first motors.

The slide driving portion 1344 receives the driving power transferred from the slide motor pack 1343 and may allow the first surgical instrument SI1 to slidably move. The first surgical instrument SI1 has one end connected to the slide driving portion 1344 and may be linearly moved by the slide driving portion 1344.

The trocar holder 1400 is arranged at one region of the translation arm 1341 and a trocar 1500 may be attached to the trocar holder 1400. In detail, the trocar holder 1400 may be arranged adjacent to the distal side end portion of the first arm third extension link 1330.

The trocar 1500 may be attached to the trocar holder 1400 and the first surgical instrument SI1 may be coupled to the trocar 1500. The first surgical instrument SI1 may be attached through the trocar 1500. Also, the first surgical instrument SI1 may be partially supported by the trocar 1500 and may be slidably moved.

The RCM point RCM1 of the first surgical instrument SI1 may be formed at one side of the trocar 1500. That is, the trocar 1500 may provide the RCM point at one side, the RCM point being a reference point of the rotation, in addition to the yaw movement and the pitch movement of the first surgical instrument SI1. When the posture of the first arm connection portion is determined, the position of the RCM point arranged on the trocar 1500 is also determined, and even when the first surgical instrument SI1 slidably moves, the position of the RCM point may be fixed.

Hereinafter, the trocar holder according to an embodiment of the present disclosure is described in detail below.

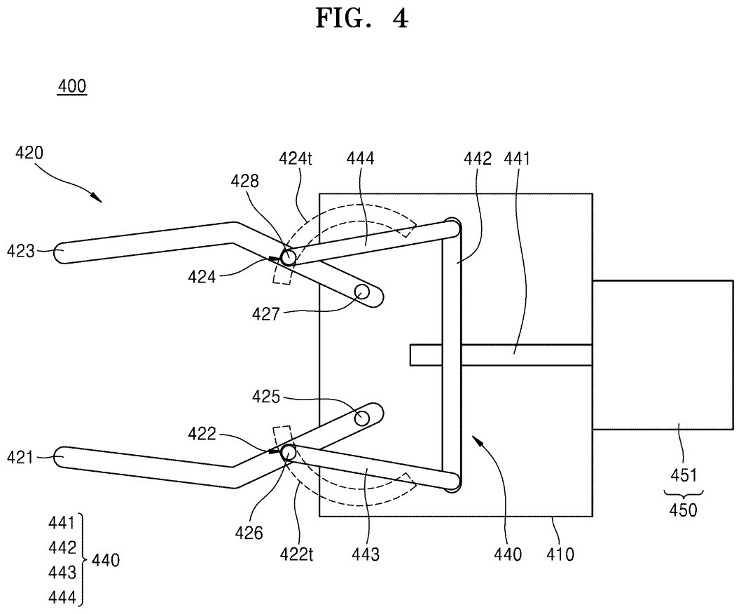

FIG. 4 is a schematic diagram of a trocar holder 400 according to an embodiment of the present disclosure.

Referring to FIG. 4, the trocar holder 400 according to an embodiment of the present disclosure may include a grip unit (or grip assembly) 420, a holder body 410, a power generation device 450, and a power transfer device 440.

The holder body 410 may be coupled to one region of the above-mentioned arm unit. In detail, the holder body 410 may be coupled to one region of a sliding arm.

The holder body 410 acts as a body of the trocar holder 400, and various components or devices included in the trocar holder 400 may be arranged on the holder body 410.

In other words, the holder body 410 may be a housing for accommodating various components or devices configuring the trocar holder 400.

In detail, the holder body 410 may accommodate at least part of the grip assembly 420 that is described later. In addition, the power transfer device 440 that is described later may be arranged in the holder body 410. Also, at least a part of the power generation device 450 that is described later may be arranged on an outside or inside of the holder body 410.

When describing the present disclosure, a part close to the arm unit is referred to as a proximal end and a part far from the arm unit is referred to as a distal end. For example, in the holder body 410, a part close to the arm unit is defined as a proximal end of the holder body 410, and a part far from the arm unit is defined as a distal end of the holder body 410. Likewise, in the grip assembly 420, a part close to the arm unit is defined as a proximal end of the grip assembly 420, and a part far from the arm unit is defined as a distal end of the grip assembly 420.

The grip assembly 420 according to an embodiment of the present disclosure may be a structure capable of gripping the trocar attached to the trocar holder 400. For example, the grip assembly may be referred to as a clamp.

The grip assembly 420 may include one or more grip portions. However, when describing the present disclosure, an example in which the grip assembly 420 includes two grip portions is described below. For example, the grip assembly 420 may include a first grip portion 421 and a second grip portion 423.

The grip assembly 420 according to an embodiment of the present disclosure may be arranged at the distal end of the holder body 410.

In detail, the first grip portion 421 and the second grip portion 423 may be at least partially accommodated in the holder body 410 described above.

In addition, the first grip portion 421 and the second grip portion 423 are arranged to face each other and may be moved to be closer to or away from each other. That is, the first grip portion 421 and the second grip portion 423 may be members that rotationally move, respectively. In detail, the first grip portion 421 may rotate about a first rotary shaft 425 and the second grip portion 423 may rotate about a second rotary shaft 427.

Here, the first rotary shaft 425 and the second rotary shaft 427 may be axially coupled to the holder body 410. In other words, rotating centers of the first grip portion 421 and the second grip portion 423 may be formed on one region of the holder body 410.

In detail, the first rotary shaft 425 is axially coupled to a proximal end of the first grip portion 421, the second rotary shaft 427 is axially coupled to a proximal end of the second grip portion 423, and the first rotary shaft 425 and the second rotary shaft 427 may be axially coupled to the holder body 410.

In an embodiment, the first rotary shaft 425 and the second rotary shaft 427 may be spaced apart from each other. In addition, the first rotary shaft 425 and the second rotary shaft 427 may be parallel to each other.

However, the concept of the present disclosure is not limited thereto, and the first rotary shaft 425 and the second rotary shaft 427 may not be parallel to each other.

In addition, the first grip portion 421 may include a first point-of-effort portion 422 which extends from the rotating center of the first grip portion 421 and receives a force for rotating the first grip portion 421 at a location spaced apart from the rotating center of the first grip portion 421.

Likewise, the second grip portion 423 may include a second point-of-effort portion 424 which extends from the rotating center of the second grip portion 423 and receives a force for rotating the second grip portion 423 at a location spaced apart from the rotating center of the second grip portion 423.

From another perspective, the point-of-effort portion (or the point-of-force portion) may be a part of the grip portion, to which the force is exerted in order to rotate the grip portion that is rotatable about the rotary shaft.

For example, a first connection member 443 that is described later may be connected to the first point-of-effort portion 422 of the first grip portion 421, and a second connection member 444 that is described later may be connected to the second point-of-effort portion 424 of the second grip portion 423. In addition, the first connection member 443 may apply the pushing or pulling force to the first point-of-effort portion 422 in order to rotate the first grip portion 421, and the second connection member 444 may apply the pushing or pulling force to the second point-of-effort portion 424 in order to rotate the second grip portion 423.

Here, the first grip portion 421 and the second grip portion 423 rotating in directions closer to each other is defined as internal rotation, and the first grip portion 421 and the second grip portion 423 rotating in directions away from each other is defined as external rotation.

That is, the first connection member 443 and the second connection member 444 respectively apply the forces to the first point-of-effort portion 422 and the second point-of-effort portion 424, so that the first grip portion 421 and the second grip portion 423 may externally or internally rotate.

Referring back to FIG. 4, a trace 422t denotes a part of a trace in which the first point-of-effort portion 422 moves, and a trace 424t denotes a part of a trace in which the second point-of-effort portion 424 moves.

In other words, when the first grip portion 421 rotates about the first rotary shaft 425, the first point-of-effort portion 422 moves along the trace 422t, and when the second grip portion 423 rotates about the second rotary shaft 427, the second point-of-effort portion 424 may move along the trace 424t.

In addition, the rotary shafts may be coupled to the first point-of-effort portion 422 and the second point-of-effort portion 424. For example, the grip assembly 420 may further include a third rotary shaft 426 and a fourth rotary shaft 428.

Here, the third rotary shaft 426 may be inserted through the first connection member 443 and the first point-of-effort portion 422 and become a rotating center of the first connection member 443. In addition, the fourth rotary shaft 428 may be inserted through the second connection member 444 and the second point-of-effort portion 424 and become a rotating center of the second connection member 444.

From another perspective, the first connection member 443 may rotate about the third rotary shaft 426, and the second connection member 444 may rotate about the fourth rotary shaft 428.

In addition, the power generation device 450 may be arranged in a region of the holder body 410. For example, as shown in the drawings, the power generation device 450 may be partially coupled to the outside of the holder body 410.

The power generation device 450 includes a driving motor 451, and the driving motor 451 may generate power for driving the grip assembly 420. Although not shown in the drawings, the power generation device 450 may further include a driving link, etc. connecting the driving motor 451 to the power transfer device 440.

In addition, the power transfer device 440 is arranged in the holder body 410 and may transfer the power generated by the driving motor 451 to the grip assembly 420.

The power transfer device 440 may include a plurality of wires, pulleys, links, joints, gears, etc.

In detail, the power transfer device 440 may include the first connection member 443 and the second connection member 444.

The first connection member 443 may be connected to the first point-of-effort portion 422 and the second connection member 444 may be connected to the second point-of-effort portion 424.

As described above, the first connection member 443 and the second connection member 444 may receive the power from the power generation device 450 and may respectively apply the force to the first point-of-effort portion 422 and the second point-of-effort portion 424.

In detail, when the first connection member 443 moves toward the proximal end side while being connected to the first point-of-effort portion 422, the first connection member 443 may apply the pulling force to the first point-of-effort portion 422 and the first grip portion 421 externally rotates. On the contrary, when the first connection member 443 moves toward the distal end side, the first connection member 443 applies the pushing force to the first point-of-effort portion 422 and the first grip portion 421 may internally rotate.

Likewise, when the second connection member 444 moves to the proximal end side while being connected to the second point-of-effort portion 424, the second connection member 444 applies the pulling force to the second point-of-effort portion 424 and the second grip portion 423 externally rotates. On the contrary, when the second connection member 444 moves toward the distal end side, the second connection member 444 applies the pushing force to the second point-of-effort portion 424 and the second grip portion 423 may internally rotate.

The power transfer device 440 may further include a third connection member 441 and a fourth connection member 442. Here, the third connection member 441 may be connected to the driving motor 451, and the fourth connection member 442 may be connected to the third connection member 441. In addition, the first connection member 443 and the second connection member 444 may be connected to the fourth connection member 442. Consequently, the power generated by the driving motor 451 may be transferred to the grip assembly 420 through the power transfer mechanism of the power transfer device 440.

Hereinafter, a trocar holder 400′ according to another embodiment of the present disclosure is described below.

Here, the trocar holder 400′ according to another embodiment of the present disclosure has a different characteristic in the rotary shaft of the grip assembly 420, as compared with the trocar holder 400 according to the embodiment described above with reference to FIG. 4. Hereinafter, different components from the above embodiment are described in detail below.

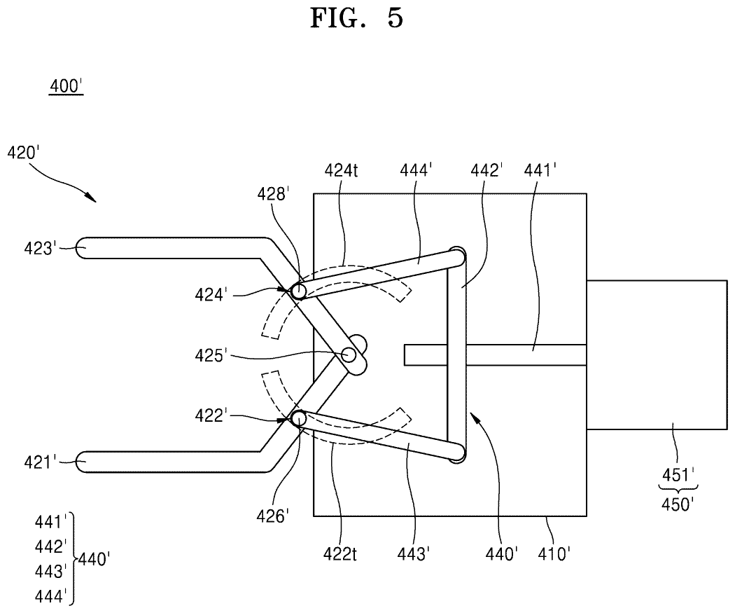

FIG. 5 is a schematic diagram of a trocar holder 400′ according to another embodiment of the present disclosure.

Referring to FIG. 5, the trocar holder 400′ according to an embodiment of the present disclosure may include a grip assembly 420′, a holder body 410′, a power generation device 450′, and a power transfer device 440′. In addition, the grip assembly 420′ may include a first grip portion 421′ and a second grip portion 423′. The power transfer device 440′ may include a first connection member 443′, a second connection member 444′, a third connection member 441′, and a fourth connection member 442′.

The holder body 410′, the power generation device 450′, the first connection member 443′, the second connection member 444′, the third connection member 441′, and the fourth connection member 442′ of the embodiment are substantially the same as the holder body 410, the power generation device 450, the first connection member 443, the second connection member 444, the third connection member 441, and the fourth connection member 442 described above with reference to FIG. 4, and thus, detailed descriptions are omitted.

In addition, the grip assembly 420′ according to the embodiment may include a first point-of-effort portion 422′, a first rotary shaft 425′, and a third rotary shaft 426′ that are related to the rotation movement of the first grip portion 421′. Also, the grip assembly 420′ may include a second point-of-effort portion 424′, the first rotary shaft 425′, and a fourth rotary shaft 428′ that are related to the rotation movement of the second grip portion 423′.

Here, the grip assembly 420′ of the trocar holder 400′ according to the embodiment is characterized in that the rotary shaft of the first grip portion 421′ and the rotary shaft of the second grip portion 423′ are the same.

In detail, the first grip portion 421′ may rotate about the first rotary shaft 425′ and the second grip portion 423′ may also rotate about the first rotary shaft 425′.

That is, the second grip portion 423′ may rotate about the rotary shaft that is coaxial with the first grip portion 421′.

The first grip portion 421′ and the second grip portion 423′ are arranged to face each other and may be moved to be closer to or away from each other. That is, the first grip portion 421′ and the second grip portion 423′ may be members that rotationally move, respectively.

Here, the first rotary shaft 425′ may be axially coupled to the holder body 410′. In other words, rotating centers of the first grip portion 421′ and the second grip portion 423′ may be formed on one region of the holder body 410′.

In detail, the first rotary shaft 425′ may be axially coupled to the proximal end of the first grip portion 421′ and the proximal end of the second grip portion 423′, and the first rotary shaft 425′ may be axially coupled to the holder body 410′.

So far, the first grip portion and the second grip portion in the grip assembly provided in the trocar holder and the first connection member and the second connection member of the power transfer device are described. The first grip portion and the second grip portion may be rotationally moved by the force transferred through the first connection member and the second connection member.

Hereinafter, the power transfer mechanism of the power transfer device is described in detail below.

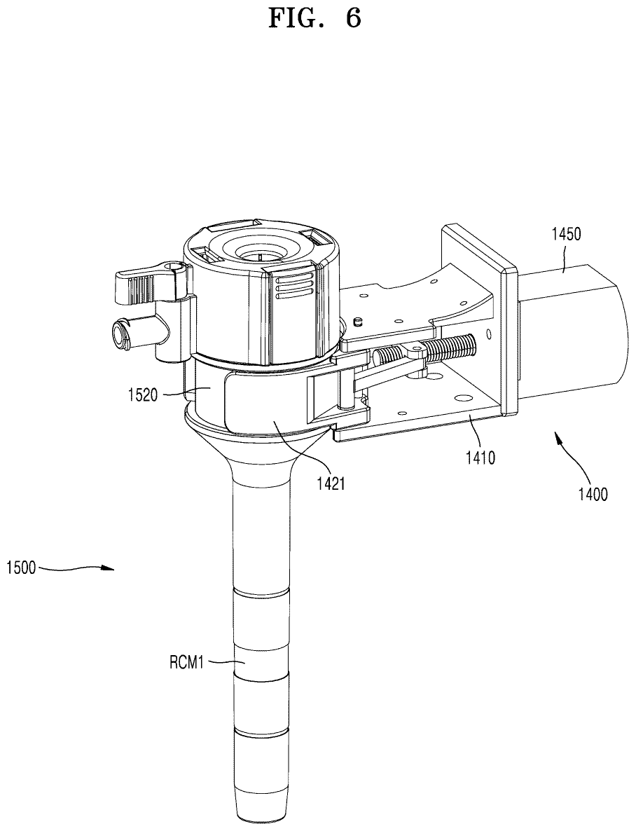

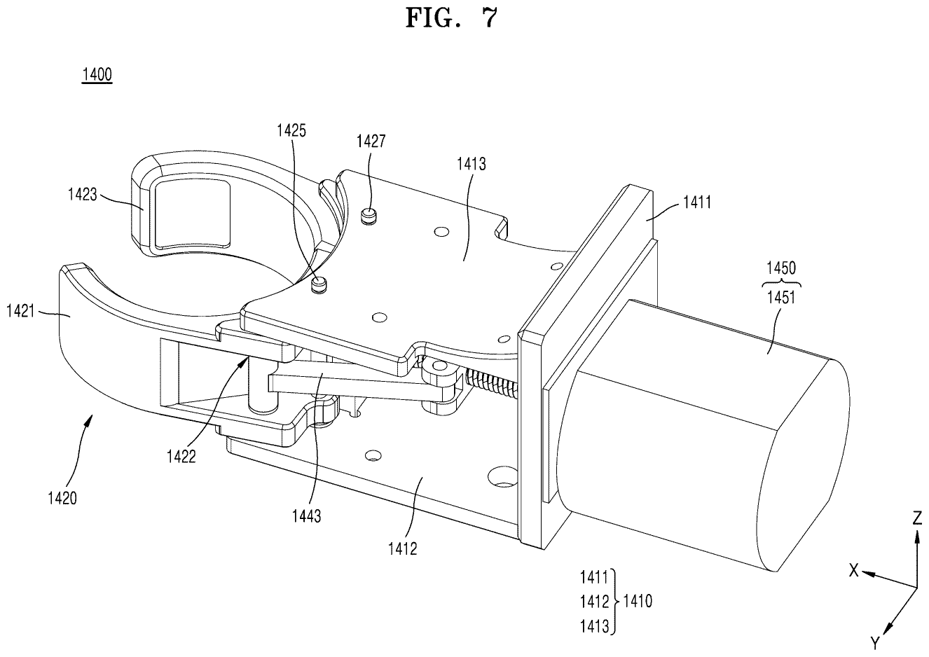

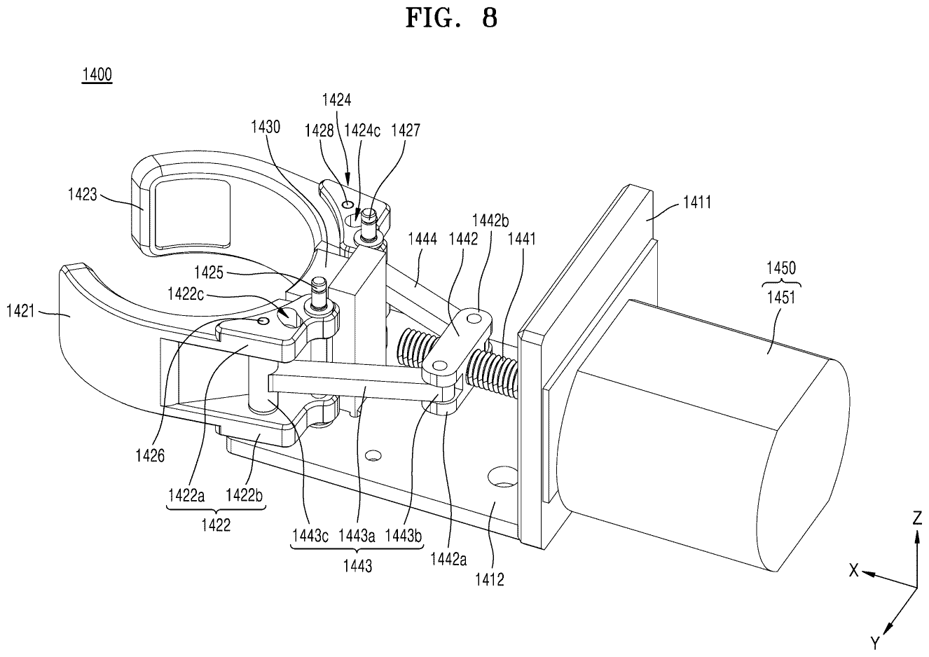

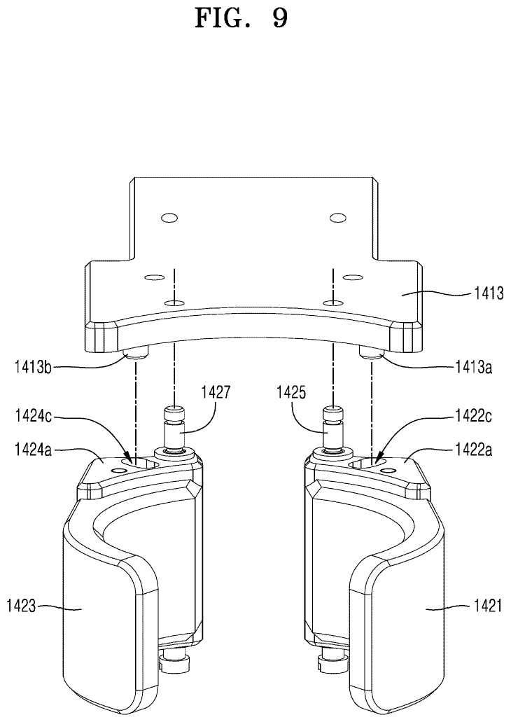

FIG. 6 is a perspective view of a trocar holder 1400 and a trocar attached to the trocar holder 1400 according to an embodiment of the present disclosure. FIG. 7 is a perspective view showing a magnified view of the trocar holder 1400 of FIG. 6. FIG. 8 is a perspective view showing a state in which an upper plate 1413 is removed from the trocar holder 1400 of FIG. 7. FIG. 9 is a diagram showing a grip assembly 1420 and the upper plate 1413 of FIG. 7.

Referring to FIGS. 6 to 9, the trocar holder 1400 according to an embodiment of the present disclosure may include the grip assembly 1420, a holder body 1410, a power generation device 1450, and a power transfer device 1440.

The holder body 1410 may be coupled to one region of the above-mentioned arm unit. In detail, the holder body 1410 may be coupled to one region of a sliding arm.

The holder body 1410 acts as a body of the trocar holder 1400, and various components or devices included in the trocar holder 1400 may be arranged on the holder body 1410.

In other words, the holder body 1410 may be a housing for accommodating various components or devices configuring the trocar holder 1400.

In detail, the holder body 1410 may accommodate at least a part of the grip assembly 1420.

In addition, the power transfer device 1440 may be arranged in the holder body 1410. Also, at least a part of the power generation device 1450 may be arranged on an outside or inside of the holder body 1410.

The holder body 1410 may include a base plate 1411, the upper plate 1413, and a lower plate 1412. In addition, although not shown in the drawings, an external housing surrounding the sides of the holder body 1410 may be provided.

The base plate 1411 may form a side wall at the proximal end side of the holder body 1410.

In addition, the upper plate 1413 and the lower plate 1412 may be coupled to one side surface of the base plate 1411. In an embodiment, the lower plate 1412 or the upper plate 1413 may be integrally formed with the base plate 1411.

The driving motor 1451 that is described later may be arranged on the base plate 1411. That is, the base plate 1411 is a coupling portion to which the driving motor 1451 is coupled and may support the driving motor 1451.

The upper plate 1413 and the lower plate 1412 are portions where the rotary shafts that are described later are coupled, and the grip assembly 1420 and the power transfer device 1440 may be supported by or fixed to the upper plate 1413 or the lower plate 1412.

The grip assembly 1420 according to an embodiment of the present disclosure may be a structure capable of gripping the trocar attached to the trocar holder 1400. In detail, the grip assembly 1420 may grip a mounting portion of the trocar.

The grip assembly 1420 according to an embodiment of the present disclosure may include a first grip portion 1421 and a second grip portion 1423.

Here, the first grip portion 1421 and the second grip portion 1423 may have inner surfaces that are curved in order to easily grip the trocar. For example, when seen on a plane, the grip portion may be formed in a hook shape. However, the concept of the present disclosure is not limited thereto, and a shape and size of the grip portion may be various.

The first grip portion 1421 and the second grip portion 1423 may be arranged at the distal end of the holder body 1410. In detail, the first grip portion 1421 and the second grip portion 1423 may be at least partially accommodated in the holder body 1410 described above.

In addition, the first grip portion 1421 and the second grip portion 1423 are arranged to face each other and may be moved to be closer to or away from each other. That is, the first grip portion 1421 and the second grip portion 1423 may be respectively members that rotationally move. In detail, the first grip portion 1421 rotates about a first rotary shaft 1425 and the second grip portion 1423 may rotate about a second rotary shaft 1427.

Here, the first rotary shaft 1425 and the second rotary shaft 1427 may be axially coupled to the holder body 1410. In other words, rotating centers of the first grip portion 1421 and the second grip portion 1423 may be formed on one region of the holder body 1410.

In detail, the first rotary shaft 1425 is axially coupled to the proximal end of the first grip portion 1421, the second rotary shaft 1427 is axially coupled to the proximal end of the second grip portion 1423, and the first rotary shaft 1425 and the second rotary shaft 1427 may be axially coupled to the lower plate 1412 and the upper plate 1413.

In an embodiment, the first rotary shaft 1425 and the second rotary shaft 1427 may be spaced apart from each other. In addition, the first rotary shaft 1425 and the second rotary shaft 1427 may be parallel to each other.

The first grip portion 1421 and the second grip portion 1423 according to an embodiment of the present disclosure are substantially the same as the first grip portion 1421 and the second grip portion 1423 described with reference to FIG. 4 within a corresponding range.

For example, the first grip portion 1421 may include a first point-of-effort portion 1422 at a location spaced apart from the first rotary shaft 1425, and the second grip portion 1423 may include a second point-of-effort portion 1424 at a location spaced apart from the second rotary shaft 1427.

In addition, the first point-of-effort portion 1422 may be a portion receiving the force for rotating the first grip portion 1421, and the second point-of-effort portion 1424 may be a portion receiving the force for rotating the second grip portion 1423.

In detail, the first point-of-effort portion 1422 may include a first point-of-effort upper end portion 1422a and a first point-of-effort lower end portion 1422b that are protruding regions. In addition, the first point-of-effort upper end portion 1422a and the first point-of-effort lower end portion 1422b are spaced apart from each other and form a space therebetween.

The first point-of-effort upper end portion 1422a and the first point-of-effort lower end portion 1422b may partially accommodate a first connection member 1443 that is described later in the space.

That is, the first connection member 1443 may be at least partially accommodated between the first point-of-effort upper end portion 1422a and the first point-of-effort lower end portion 1422b.

In addition, a third rotary shaft 1426 may be inserted through the first point-of-effort upper end portion 1422a and the first point-of-effort lower end portion 1422b and become a rotating center of the first connection member 1443. In detail, the third rotary shaft 1426 may not be coupled to the holder body 1410. That is, the third rotary shaft 1426 may be moved along with the first grip portion 1421 while being inserted in the first grip portion 1421. From another perspective, the third rotary shaft 1426 may rotate about the first rotary shaft 1425 while being arranged parallel to the first rotary shaft 1425.

Also, in an embodiment, a movable coupling hole may be formed in one side of the proximal end of the first grip portion 1421. In detail, at least one of the first point-of-effort upper end portion 1422a and the first point-of-effort lower end portion 1422b described above may have the movable coupling hole.

In detail, a first movable coupling hole 1422c may be formed in the first point-of-effort upper end portion 1422a. Referring to FIGS. 8 and 9, the first movable coupling hole 1422c may be a recess formed to a certain depth in the first point-of-effort upper end portion 1422a. In addition, the first movable coupling hole 1422c may be formed along a trace in which the first grip portion 1421 is rotated.

As shown in FIG. 9, a first protrusion 1413a may be formed at the proximal end side of the upper plate 1413. In detail, the first protrusion 1413a may be formed at a position corresponding to the first movable coupling hole 1422c.

The first protrusion 1413a may be inserted into the first movable coupling hole 1422c, and the first protrusion 1413a may be relatively moved in the first movable coupling hole 1422c while being inserted into the first movable coupling hole 1422c.

In detail, the first protrusion 1413a is fixed to the upper plate 1413, and thus, the first grip portion 1421 may be rotated within a range in which the first movable coupling hole 1422c does not interfere with the first protrusion 1413a when the first grip portion 1421 is rotated.

That is, a range of a rotation radius of the first grip portion 1421 may be defined by the first movable coupling hole 1422c and the first protrusion 1413a.

In an embodiment, a movable coupling hole may be formed in the first point-of-effort lower end portion 1422b and a corresponding protrusion may be formed on the lower plate 1412.

Likewise, the second point-of-effort portion 1424 of the second grip portion 1423 may include a second point-of-effort upper end portion 1424a and a second point-of-effort lower end portion (not shown), a fourth rotary shaft 1428, and a second movable coupling hole 1424c. In addition, the upper plate 1413 may have a second protrusion 1413b corresponding to the second movable coupling hole 1424c.

The second point-of-effort upper end portion 1424a and the second point-of-effort lower end portion (not shown), the fourth rotary shaft 1428, the second movable coupling hole 1424c, and the second protrusion 1413b are substantially the same as the above-mentioned first point-of-effort upper end portion 1422a, the first point-of-effort lower end portion 1422b, the third rotary shaft 1426, the first movable coupling hole 1422c, and the first protrusion 1413a, and thus, detailed descriptions are omitted.

In addition, the power generation device 1450 may include the driving motor 1451. The driving motor 1451 may generate power for driving the grip assembly 1420. In an embodiment, the driving motor 1451 may be arranged on the base plate 1411. For example, as shown in the drawings, the driving motor 1451 may be partially coupled to the outside of the holder body 1410.

Although not shown in the drawings, the power generation device 1450 may further include a driving link (not shown), etc. connecting the driving motor 1451 to the power transfer device 1440.

In addition, the power transfer device 1440 is arranged in the holder body 1410 and may transfer the power generated by the driving motor 1451 to the grip assembly 1420.

In detail, the power transfer device 1440 may include one shaft (or a power transfer shaft) 1441, a linear movement portion 1442, the first connection member 1443, and a second connection member 1444.

The one shaft 1441 may be connected to the power generation device 1450 and may define a power transfer axis AX1. Although it is shown in the drawings that the one shaft 1441 is directly connected to the driving motor 1451, the concept of the present disclosure is not limited thereto, and the one shaft 1441 may be connected to a driving link of the power generation device 1450 and may receive the power through the driving link.

The one shaft 1441 may rotate about a rotary shaft in the longitudinal direction. That is, the one shaft 1441 may rotate about the power transfer axis AX1.

The one shaft 1441 may have a screw thread on the outer circumference. For example, the one shaft 1441 may correspond to a ball screw or a lead screw.

The linear movement portion 1442 may be a member that performs translational movement between the proximal end and the distal end along the power transfer axis AX1. In other words, the linear movement portion 1442 may perform the translational movement between the proximal end and the distal end of the one shaft 1441 along the one shaft 1441.

In detail, the linear movement portion 1442 may perform the translational movement as the one shaft 1441 rotates about the power transfer axis AX1.

The linear movement portion 1442 according to an embodiment of the present disclosure may include a nut portion (not shown), a first hinge portion 1442a, and a second hinge portion 1442b.

The nut portion is a portion through which the one shaft 1441 is inserted and may have a screw recess in the inner circumference. Therefore, the screw recess formed in the inner circumference of the nut portion may be engaged with the screw thread formed on the outer circumference of the one shaft 1441.

In an embodiment, the nut portion may include a ball nut. The ball nut may include a ball circulating along the screw recess.

The first hinge portion 1442a and the second hinge portion 1442b may be provided on both opposite sides of the nut portion. For example, the first hinge portion 1442a and the second hinge portion 1442b may be arranged to face each other.

The first hinge portion 1442a may be connected to one end portion of the first connection member 1443. In detail, the first hinge portion 1442a may be coupled to the first connection member 1443 in a hinged manner via a hinge pin 1443d.

Likewise, the second hinge portion 1442b may be connected to one end portion of the second connection member 1444. In detail, the second hinge portion 1442b may be coupled to the second connection member 1444 in a hinged manner via a hinge pin 1444d.

Therefore, the first connection member 1443 and the second connection member 1444 may be rotatably coupled to the linear movement portion 1442.

In addition, the first connection member 1443 may be a connection member connecting between the first grip portion 1421 and the linear movement portion 1442. In detail, one end portion 1443b of the first connection member 1443 may be rotatably coupled to the first hinge portion 1442a, and the other end portion 1443c of the first connection member 1443 may be rotatably coupled to the first grip portion 1421. In detail, one end portion 1443b of the first connection member 1443 may be hinge-coupled to the first hinge portion 1442a via the hinge pin 1443d, and the other end portion 1443c of the first connection member 1443 may be axially coupled to the first point-of-effort upper end portion 1422a and the first point-of-effort lower end portion 1422b via the third rotary shaft 1426. Here, the other end portion 1443c of the first connection member 1443 may be formed as a cylindrical shape surrounding the third rotary shaft 1426.

The second connection member 1444 may be a connection member connecting between the second grip portion 1423 and the linear movement portion 1442. In detail, one end portion of the second connection member 1444 may be rotatably coupled to the second hinge portion 1442b, and the other end portion of the second connection member 1444 may be rotatably coupled to the second grip portion 1423. In detail, one end portion of the second connection member 1444 may be hinge-coupled to the second hinge portion 1442b via the hinge pin 1444d, and the other end portion of the second connection member 1444 may be axially coupled to the second point-of-effort upper end portion 1424a and the second point-of-effort lower end portion (not shown) via the fourth rotary shaft 1428. Here, the other end portion of the second connection member 1444 may be formed as a cylindrical shape surrounding the fourth rotary shaft 1428.

Hereinafter, in the trocar holder 1400 according to an embodiment of the present disclosure, the mechanism of the power transfer device 1440 that transfers the power of the driving motor 1451 to the grip assembly 1420 is described.

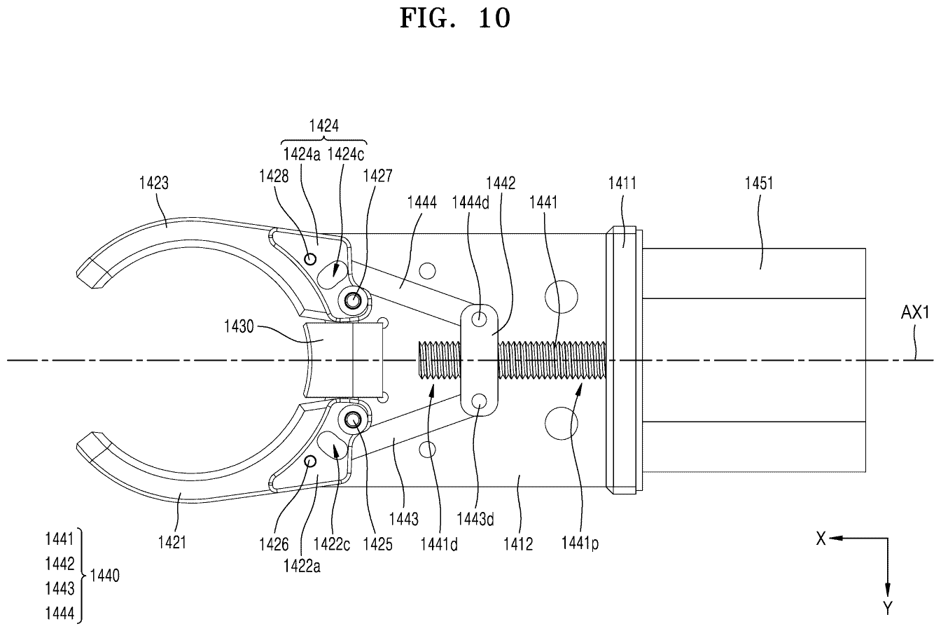

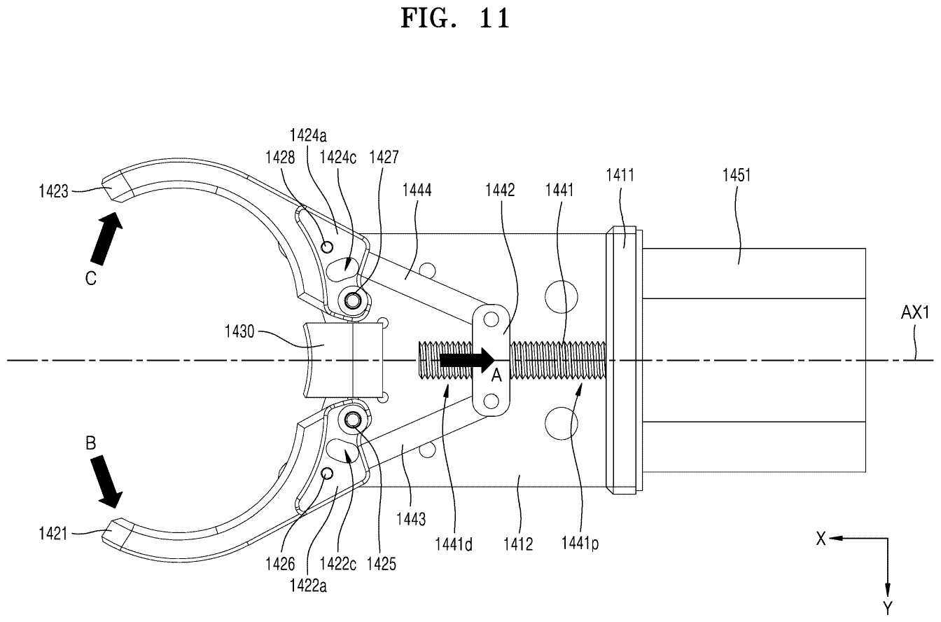

FIG. 10 is a plan view of the trocar holder 1400 of FIG. 8, and FIG. 11 is a plan view for illustrating an opening operation of the trocar holder 1400 of FIG. 10. In other words, FIG. 10 shows a closed state of the grip assembly 1420, and FIG. 11 shows an opened state of the grip assembly 1420.

The one shaft 1441 is rotated about the power transfer axis AX1 due to the operation of the driving motor 1451, and accordingly, the linear movement portion 1442 may perform the translational movement toward the proximal end side. That is, the linear movement portion 1442 may be moved in a direction of arrow A.

Accordingly, the first connection member 1443 and the second connection member 1444 connected to the linear movement portion 1442 are moved toward the proximal end side.

When the first connection member 1443 is moved toward the proximal end side while being connected to the first point-of-effort upper end portion 1422a and the first point-of-effort lower end portion 1422b, the first connection member 1443 applies a pulling force to the first point-of-effort portion 1422, and the first grip portion 1421 is rotated in a direction of arrow B.

That is, the first grip portion 1421 externally rotates.

Likewise, when the second connection member 1444 moves toward the proximal end side while being connected to the second point-of-effort upper end portion and the second point-of-effort lower end portion, the second connection member 1444 applies a pulling force to the second point-of-effort portion 1424, and the second grip portion 1423 is rotated in a direction of arrow C. That is, the second grip portion 1423 externally rotates.

As described above, the first grip portion 1421 and the second grip portion 1423 are spaced apart from each other due to the operation of the driving motor 1451, and then, an opening operation may be performed.

In addition, when the one shaft 1441 rotates in the opposite direction, the linear movement portion 1442 may perform the translational movement toward the distal end side. In addition, the first connection member 1443 and the second connection member 1444 connected to the linear movement portion 1442 are moved toward the distal end side.

Accordingly, the first connection member 1443 applies a pushing force to the first point-of-effort portion 1422, and thus, the grip portion 1421 may internally rotate. In addition, the second connection member 1444 applies a pushing force to the second point-of-effort portion 1424, and the second grip portion 1423 may internally rotate. That is, the grip assembly 1420 may be closed.

As described above, the first grip portion 1421 and the second grip portion 1423 are rotated to be closer to each other due to the operation of the driving motor 1451, and then, a closing operation may be performed.

In addition, the trocar holder 1400 according to an embodiment of the present disclosure may further include a trocar sensing unit 1430.

Here, the trocar sensing unit 1430 is a device sensing the trocar attached to the trocar holder 1400.

In detail, the trocar sensing unit 1430 may sense the trocar when the trocar approaches or physically comes into contact with it. For example, the trocar sensing unit 1430 may include a physical switch on the outside, the physical switch being operated by a pressure. In addition, when the mounting portion of the trocar comes into contact with the trocar sensing unit 1430 and the switch is pressed, the trocar may be sensed.

In an embodiment, the trocar sensing unit 1430 may be arranged at the distal end of the holder body 1410. In detail, the trocar sensing unit 1430 may be arranged between the rotary shaft of the first grip portion 1421 and the rotary shaft of the second grip portion 1423. That is, the trocar sensing unit 1430 may be arranged so as to easily sense the trocar when the trocar is located between the first grip portion 1421 and the second grip portion 1423.

In detail, the trocar sensing unit 1430 may be arranged on an extension from the power transfer axis AX1. However, the concept of the present disclosure is not limited thereto, and the trocar sensing unit 1430 may be arranged at a position where the trocar may be sensed, in or out of the holder body 1410.

In the trocar holder 1400 according to an embodiment of the present disclosure, when the trocar sensing unit 1430 senses the trocar, the driving motor 1451 may operate. In detail, the driving motor 1451 may be operated according to a signal provided by the trocar sensing unit 1430 sensing the trocar.

Accordingly, the grip assembly 1420 in an opened state may be automatically closed when receiving the power from the driving motor 1451. That is, the trocar holder 1400 according to an embodiment of the present disclosure may sense whether the trocar is appropriately located through the trocar sensing unit 1430 and drive the driving motor 1451 to attach the trocar only through an operator's manipulation of appropriately locating the trocar without using an additional lever or handle.

Through the trocar holder 1400 according to an embodiment of the present disclosure, the operator may easily attach the trocar to a surgical robot, and may reduce an RCM point of the trocar escaping from an appropriate position due to unskilled manipulation.

Hereinafter, a trocar holder 2400 according to another embodiment of the present disclosure is described below. The trocar holder 2400 according to another embodiment of the present disclosure differs from the trocar holder 1400 described above with reference to FIG. 7, in view of a power transfer device 2440. Hereinafter, different components from the above embodiment are described in detail below.

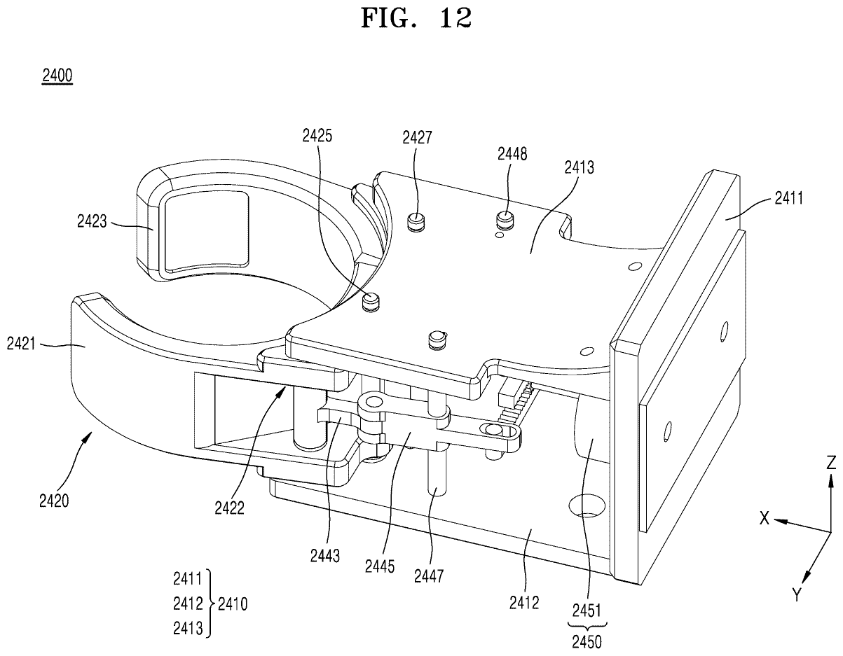

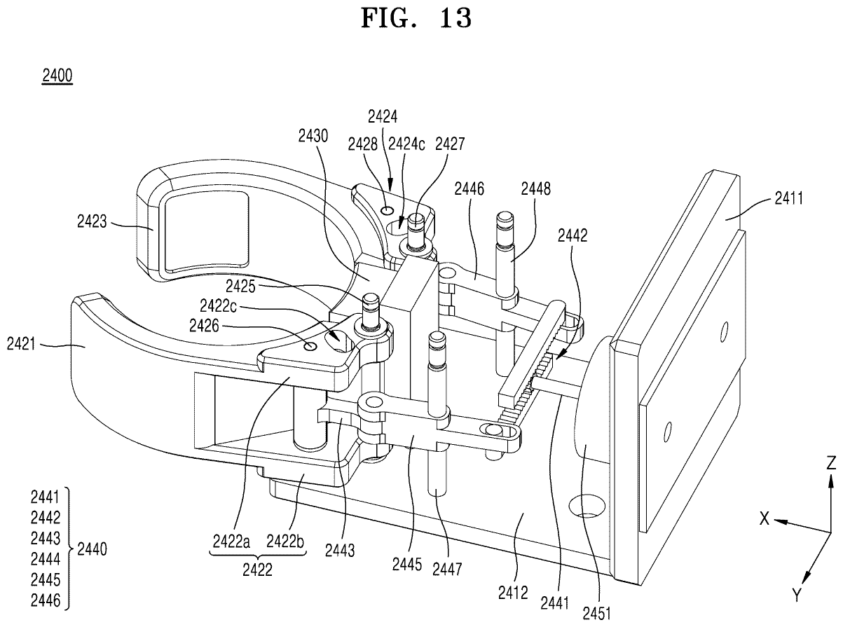

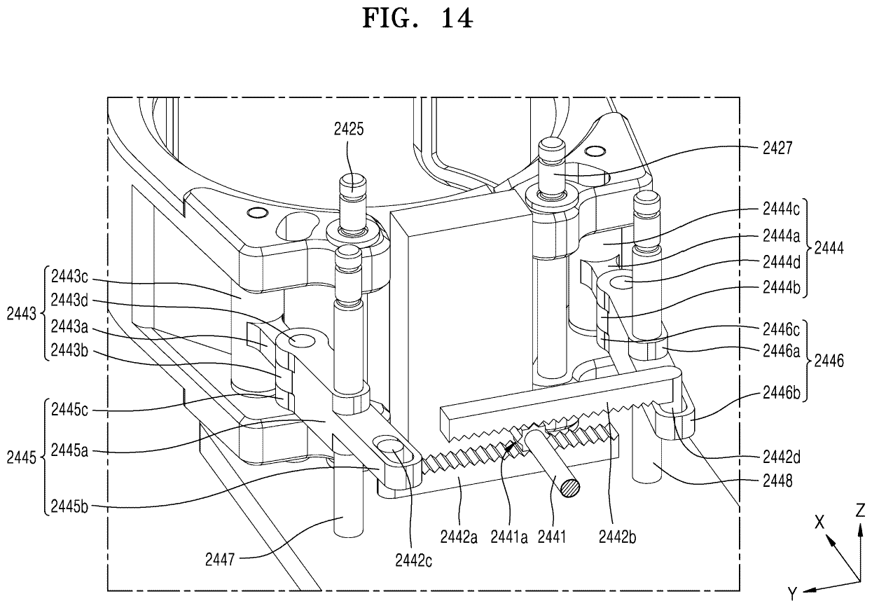

FIG. 12 is a perspective view of the trocar holder 2400 according to another embodiment of the present disclosure. FIG. 13 is a perspective view showing a state in which an upper plate 2413 is removed from the trocar holder 2400 of FIG. 12. FIG. 14 is a perspective view showing some components of the trocar holder 2400 of FIG. 13 in a different angle.

Referring to FIGS. 12 to 14, the trocar holder 2400 according to another embodiment of the present disclosure may include a grip assembly 2420, a holder body 2410, a power generation device 2450, and a power transfer device 2440. In addition, the grip assembly 2420 includes a first grip portion 2421 and a second grip portion 2423, and the holder body 2410 may include a base plate 2411, an upper plate 2413, and a lower plate 2412. The power generation device 2450 may include a driving motor 2451.

The first grip portion 2421, the second grip portion 2423, the base plate 2411, the upper plate 2413, the lower plate 2412, and the driving motor 2451 are substantially the same as the first grip portion 1421, the second grip portion 1423, the base plate 1411, the upper plate 1413, the lower plate 1412, and the driving motor 1451 described above with reference to FIG. 7, and thus, detailed descriptions are omitted.

In addition, the power transfer device 2440 may include one shaft (or a power transfer shaft) 2441, a first rack 2442a, a second rack 2442b, a first lever member 2445, a second lever member 2446, a first connection member 2443, and a second connection member 2444.

Here, the one shaft 2441 may be connected to the power generation device 2450 and may define the power transfer axis AX1.

Although the one shaft 2441 is directly connected to the driving motor 2451 in the drawings, the concept of the present disclosure is not limited thereto, and the one shaft 2441 may be connected to a driving link of the power generation device 2450 and may receive the power through the driving link.

The one shaft 2441 may rotate about a rotary shaft in the longitudinal direction. That is, the one shaft 2441 may rotate about the power transfer axis AX1.

Also, the one shaft 2441 may include a pinion gear 2441a on one region. For example, the one shaft 2441 may include the pinion gear 2441a at an end portion of the distal end side.

In addition, the first rack 2442a and the second rack 2442b may be arranged to face each other with the pinion gear 2441a interposed therebetween. Also, the first rack 2442a and the second rack 2442b may be arranged to cross or intersect the power transfer axis AX1 of the one shaft 2441.

From another perspective, the first rack 2442a may be arranged on a virtual plane that is perpendicular to a first rotary shaft 2425 and a second rotary shaft 2427. The second rack 2442b may be arranged on another virtual plane that is perpendicular to the first rotary shaft 2425 and the second rotary shaft 2427. In detail, the first rack 2442a and the second rack 2442b may be arranged in parallel with a Y-axis of the drawing.

From another perspective, based on a virtual plane that is perpendicular to the first rotary shaft 2425 and the second rotary shaft 2427 and includes the power transfer axis AX1 of the one shaft 2441, the first rack 2442a may be arranged at one side and the second rack 2442b may be arranged at the other side.

The first rack 2442a and the second rack 2442b may each include, on one surface, a gear that is engaged with the pinion gear 2441a. In other words, the first rack 2442a and the second rack 2442b may have gears on facing surfaces.

Also, the first rack 2442a may include a first protrusion 2442c on one end portion. Here, the first protrusion 2442c may be formed to be inserted into a first elongated hole 2445d of the first lever member 2445 that is described later.

Likewise, the second rack 2442b may include a second protrusion 2442d at one end portion. Here, the second protrusion 2442d may be formed to be inserted into a second elongated hole 2446d of a second lever member 2446 that is described later.

The first lever member 2445 may connect the first connection member 2443 to the first rack 2442a, and the second lever member 2446 may connect the second connection member 2444 to the second rack 2442b.

That is, one end portion 2445b of the first lever member 2445 may be connected to the first rack 2442a, and the other end portion 2445c of the first lever member 2445 may be connected to the first connection member 2443.

In detail, the first lever member 2445 may rotate about a fifth rotary shaft 2447. Here, the fifth rotary shaft 2447 may be coupled to the upper plate 2413 and the lower plate 2412. In addition, the fifth rotary shaft 2447 may be inserted through a body 2445a of the first lever member 2445 so as to form a rotating center of the first lever member 2445.

One end portion 2445b of the first lever member 2445, which extends from the rotating center of the first lever member 2445, is connected to the first rack 2442a, and the other end portion 2445c of the first lever member 2445, which extends from the rotating center of the first lever member 2445, may be hinge-coupled to the first connection member 2443.

In detail, the first elongated hole 2445d may be formed in one end portion 2445b of the first lever member 2445. In addition, while the first protrusion 2442c of the first rack 2442a is inserted into the first elongated hole 2445d, the first protrusion 2442c may be formed to move a certain degree within the first elongated hole 2445d.

One end portion 2446b of the second lever member 2446 may be connected to the second rack 2442b and the other end portion 2446c of the second lever member 2446 may be connected to the second connection member 2444.

In detail, the second lever member 2446 may rotate about a sixth rotary shaft 2448. Here, the sixth rotary shaft 2448 may be coupled to the upper plate 2413 and the lower plate 2412. In addition, the sixth rotary shaft 2448 may be inserted through a body 2446a of the second lever member 2446 so as to form a rotating center of the second lever member 2446.

One end portion 2446b of the second lever member 2446, which extends from the rotating center of the second lever member 2446, is connected to the second rack 2442b, and the other end portion 2446c of the second lever member 2446, which extends from the rotating center of the second lever member 2446, may be hinge-coupled to the second connection member 2444.

In detail, the second elongated hole 2446d may be formed in one end portion 2446b of the second lever member 2446. In addition, while the second protrusion 2442d of the second rack 2442b is inserted into the second elongated hole 2446d, the second protrusion 2442d may be formed to move a certain degree within the second elongated hole 2446d.

In addition, the first connection member 2443 may connect the first lever member 2445 to the first grip portion 2421.

Here, a connecting structure of the first connection member 2443 to the first grip portion 2421 is substantially the same as the first connection member 1443 in the embodiment described above with reference to FIG. 7, and thus, detailed descriptions are omitted.

An end portion at the proximal end side of the first connection member 2443 may be hinge-coupled to an end portion at the distal end side of the first lever member 2445. That is, the first connection member 2443 and the first lever member 2445 may be rotatably coupled.

Here, the hinge pin 2443d that connects the first connection member 2443 to the first lever member 2445 to be rotatable may be arranged parallel to the fifth rotary shaft 2447. Also, the hinge pin 2443d may be arranged parallel to the third rotary shaft 2426. In other words, the third rotary shaft 2426, the fifth rotary shaft 2447, and the hinge pin 2443d may be arranged parallel to one another.

The second connection member 2444 may connect the second lever member 2446 to the second grip portion 2423.

Here, a connecting structure of the second connection member 2444 to the second grip portion 2423 is substantially the same as the second connection member 1444 in the embodiment described above with reference to FIG. 7, and thus, detailed descriptions are omitted.

An end portion at the proximal end side of the second connection member 2444 may be hinge-coupled to an end portion at the distal end side of the second lever member 2446. That is, the second connection member 2444 and the second lever member 2446 may be rotatably coupled.

Here, the hinge pin 2444d that couples the second connection member 2444 to the second lever member 2446 to be rotatable may be arranged parallel to the sixth rotary shaft 2448. Also, the hinge pin 2444d may be arranged parallel to the fourth rotary shaft 2428. In other words, the fourth rotary shaft 2428, the sixth rotary shaft 2448, and the hinge pin 2444d may be arranged parallel to one another.

Hereinafter, in the trocar holder 2400 according to another embodiment of the present disclosure, the mechanism of the power transfer device 2440 that transfers the power of the driving motor 2451 to the grip assembly 2420 is described.

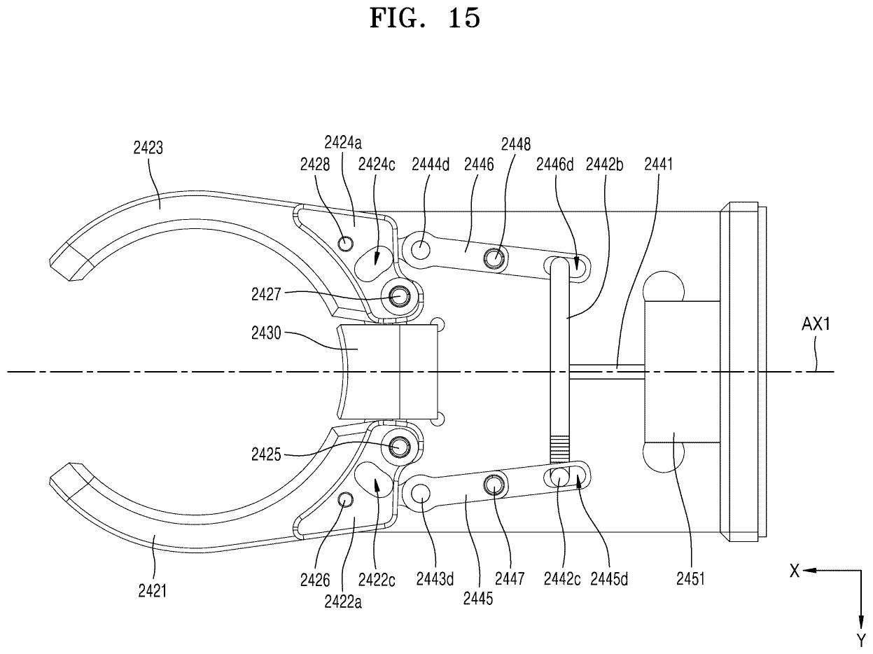

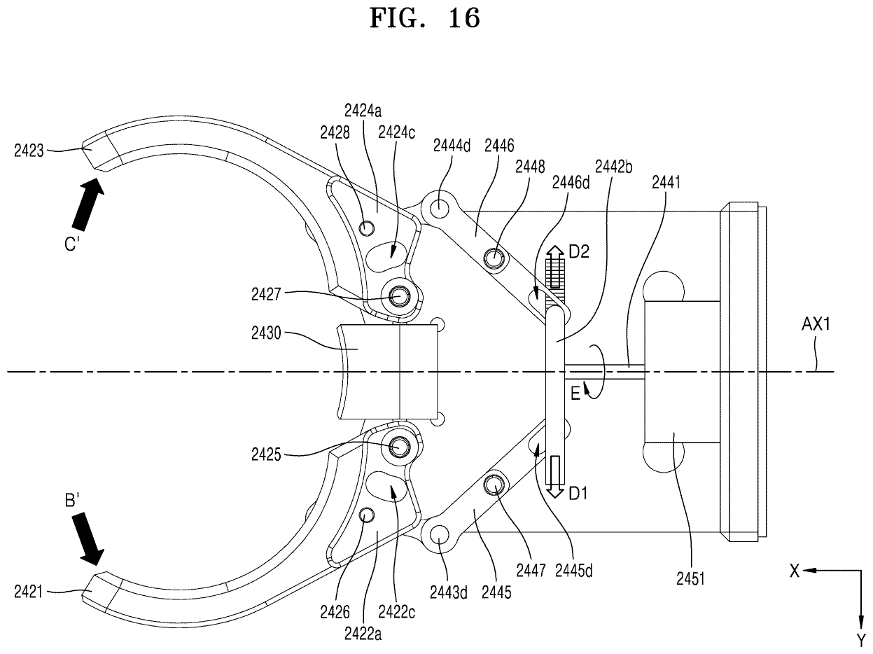

FIG. 15 is a plan view of the trocar holder 2400 of FIG. 13, and FIG. 16 is a plan view for illustrating an opening operation of the trocar holder 2400 of FIG. 15. In other words, FIG. 15 shows a closed state of the grip assembly 2420, and FIG. 16 shows an opened state of the grip assembly 2420.

The one shaft 2441 is rotated about the power transfer axis AX1 due to the operation of the driving motor 2451, and the pinion gear 2441a provided on one region of the one shaft 2441 may be also rotated.

Accordingly, the first rack 2442a and the second rack 2442b arranged to be engaged with the pinion gear 2441a may be linearly moved.

Here, as the pinion gear 2441a rotates in one direction, the first rack 2442a and the second rack 2442b may be moved in opposite directions to each other.

From another perspective, the first rack 2442a may be moved in the longitudinal direction of the first rack 2442a and the second rack 2442b may be moved in the longitudinal direction of the second rack 2442b due to the rotation of the one shaft 2441 in one direction, so that one end portion of the first rack 2442a and one end portion of the second rack 2442b may be closer to or away from each other.

In detail, when one end portion of the first rack 2442a, which is connected to the first lever member 2445, and one end portion of the second rack 244b, which is connected to the second lever member 2446, are closer to each other due to the rotation of the one shaft 2441 in one direction, an opening operation in which the first grip portion 2421 and the second grip portion 2423 are away from each other may be performed.

In other words, the first rack 2442a may be moved in a direction of arrow D2 and the second rack 2442b may be moved in a direction of arrow D1 due to the rotation of the one shaft 2441 in one direction. In addition, due to the linear movements of the first rack 2442a and the second rack 2442b, the first grip portion 2421 may externally rotate in a direction of arrow B′ and the second grip portion 2423 may externally rotate in a direction of arrow C′.

In detail, when the first rack 2442a is moved in the direction of arrow D2, the first protrusion 2442c of the first rack 2442a may slidably move toward the end portion within the first elongated hole 2445d. Accordingly, the first lever member 2445 may rotate so that one end portion 2445b at the proximal end side of the first lever member 2445 approaches the one shaft 2441 and the other end portion 2445c is away from the one shaft 2441.

According to the rotation of the first lever member 2445 as above, the first connection member 2443 connected to the other end portion 2445c of the first lever member 2445 may receive the pulling force toward the proximal end side, and then, the first grip portion 2421 may externally rotate.

Likewise, when the second rack 2442b is moved in the direction of arrow D1, the second protrusion 2442d of the second rack 2442b may slidably move toward the end portion within the second elongated hole 2446d. Accordingly, the second lever member 2446 may rotate so that one end portion 2446b at the proximal end side of the second lever member 2446 approaches the one shaft 2441 and the other end portion 2446c is away from the one shaft 2441.

According to the rotation of the second lever member 2446 as above, the second connection member 2444 connected to the other end portion 2446c of the second lever member 2446 may receive the pulling force toward the proximal end side, and then, the second grip portion 2423 may externally rotate.

As described above, an opening operation in which the first grip portion 2421 and the second grip portion 2423 are away from each other due to the operation of the driving motor 2451 may be performed.

On the contrary, when one end portion of the first rack 2442a, which is connected to the first lever member 2445, and one end portion of the second rack 2442b, which is connected to the second lever member 2446, are away from each other due to the rotation of the one shaft 2441 in one direction, a closing operation in which the first grip portion 2421 and the second grip portion 2423 approach each other may be performed.

That is, when the first rack 2442a moves in the direction of arrow D1 and the second rack 2442b moves in the direction of arrow D2, one end portion 2445b of the first lever member 2445 and one end portion 2446b of the second lever member 2446 are rotated to be away from the one shaft 2441, respectively. Accordingly, the first connection member 2443 and the second connection member 2444 may respectively apply the pushing force to the first grip portion 2421 and the second grip portion 2423, and thus, the first grip portion 2421 and the second grip portion 2423 may internally rotate.

As described above, due to the operation of the driving motor 2451, the closing operation in which the first grip portion 2421 and the second grip portion 2423 are closer to each other may be performed.

The trocar holder 2400 according to another embodiment of the present disclosure may further include a trocar sensing unit 2430. Therefore, as described above, the trocar holder 2400 may operate the driving motor 2451 when the trocar sensing unit 2430 senses or detects the trocar. In detail, the driving motor 2451 may be operated according to a signal provided by the trocar sensing unit 2430 upon sensing the trocar.

Accordingly, the grip assembly 2420 in an opened state may be automatically closed when receiving the power from the driving motor 2451. That is, the trocar holder 2400 according to an embodiment of the present disclosure may sense whether the trocar is appropriately located through the trocar sensing unit 2430 and drive the driving motor 2451 to attach the trocar only through an operator's manipulation of appropriately locating the trocar without using an additional lever or handle.

According to the present disclosure as above, the trocar fastening method of the trocar holder in a surgical robot may be automated so as to easily fasten the trocar and may reduce errors that may occur during manipulation.

While the present disclosure has been particularly shown and described with reference to preferred embodiments thereof, it will be understood by those skilled in the art that various changes in form and details may be made therein without departing from the spirit and scope of the present disclosure as defined by the appended claims. The preferred embodiments should be considered in descriptive sense only and not for purposes of limitation. Therefore, the concept of the present disclosure is defined not by the detailed description of the present disclosure but by the appended claims, and all differences within the scope will be construed as being included in the present disclosure.

What is claimed is:

1. A trocar holder of a surgical robot, the trocar holder comprising:
a grip assembly comprising a first grip portion and a second grip portion that are arranged to face each other and configured to move closer to or away from each other;
a holder body to which at least a part of the grip assembly is connected;
a power generation device including a driving motor for generating power for driving the grip assembly; and
a power transfer device disposed in the holder body and configured to transfer the power generated by the driving motor to the grip assembly.

2. The trocar holder of claim 1, wherein,
when a driving shaft of the driving motor is rotated in a first direction, the first grip portion and the second grip portion are rotated in a direction approaching each other, and
when the driving shaft is rotated in a second direction opposite to the first direction, the first grip portion and the second grip portion are rotated in a direction away from each other.

3. The trocar holder of claim 1, wherein
the power transfer device is configured to allow the first grip portion and the second grip portion to rotate in opposite directions while being arranged symmetrically with each other.

4. The trocar holder of claim 1, wherein
the first grip portion and the second grip portion are rotated coaxially.

5. The trocar holder of claim 1, wherein
the first grip portion rotates about a first rotary shaft, and the second grip portion rotates about a second rotary shaft spaced apart from the first rotary shaft.

6. The trocar holder of claim 5, wherein
the first rotary shaft is axially coupled to a proximal end of the first grip portion and the holder body, and the second rotary shaft is axially coupled to a proximal end of the second grip portion and the holder body.

7. The trocar holder of claim 1, further comprising a trocar sensor configured to sense a trocar attached to the trocar holder.

8. The trocar holder of claim 7, wherein the driving motor is configured to automatically operate based on a signal provided by the trocar sensor when the trocar sensor senses the trocar.

9. The trocar holder of claim 7, wherein
the trocar sensor is disposed between a first rotary shaft of the first grip portion and a second rotary shaft of the second grip portion.

10. The trocar holder of claim 1, wherein
the first grip portion includes a first point-of-force portion extending from a first rotating center of the first grip portion and configured to receive a force for rotating the first grip portion at a position spaced apart from the first rotating center of the first grip portion, and
the second grip portion includes a second point-of-force portion extending from a second rotating center of the second grip portion and configured to receive a force for rotating the second grip portion at a position spaced apart from the second rotating center of the second grip portion.

11. The trocar holder of claim 10, wherein the power transfer device includes a first connection member connected to the first point-of-force portion and a second connection member connected to the second point-of-force portion.

12. The trocar holder of claim 11, wherein
the first connection member and the second connection member receive the power from the power generation device and apply force to the first point-of-force portion and the second point-of-force portion, respectively, and allow the first grip portion and the second grip portion to rotate externally or internally.

13. The trocar holder of claim 1, wherein
at least one movable coupling hole is defined in a region on a proximal end of each of the first grip portion and the second grip portion, and
protrusions are disposed on a first plate of the holder body, and are configured to be inserted into the at least one movable coupling hole and relatively move in the at least one movable coupling hole.

14. The trocar holder of claim 1, wherein the power transfer device includes:
one shaft connected to the power generation device and defining a power transfer axis;
a linear movement portion configured to perform translational movement between a proximal end and a distal end along the power transfer axis;
a first connection member connecting the linear movement portion to the first grip portion; and
a second connection member connecting the linear movement portion to the second grip portion.

15. The trocar holder of claim 14, wherein the first grip portion and the second grip portion are configured to:
perform an opening operation by moving away from each other when the linear movement portion moves toward a proximal end, and
perform a closing operation by moving closer to each other when the linear movement portion moves toward a distal end.

16. The trocar holder of claim 14, wherein
the linear movement portion performs translational movement when the one shaft rotates about the power transfer axis.

17. The trocar holder of claim 14, wherein the linear movement portion comprises:
a nut portion through which the one shaft is inserted; and
a first hinge portion and a second hinge portion disposed on both sides of the nut portion,
wherein one end portion of the first connection member is rotatably coupled to the first hinge portion and another end portion of the first connection member is rotatably coupled to the first grip portion, and
wherein one end portion of the second connection member is rotatably coupled to the second hinge portion and another end portion of the second connection member is rotatably coupled to the second grip portion.

18. The trocar holder of claim 1, wherein
the power transfer device comprises:
one shaft including a pinion gear, connected to the power generation device, and defining a power transfer axis;
a first rack and a second rack that are arranged to face each other with the pinion gear interposed therebetween, each of the first rack and the second rack including a gear engaged with the pinion gear;
a first lever member connected to the first rack;
a second lever member connected to the second rack;
a first connection member connecting the first lever member to the first grip portion; and
a second connection member connecting the second lever member to the second grip portion.

19. The trocar holder of claim 18, wherein
the first lever member has one end portion extending from a first rotating center of the first lever member and connected to the first rack, and another end portion extending from the first rotating center of the first lever member and hinge-coupled to the first connection member, and
the second lever member has one end portion extending from a second rotating center of the second lever member and connected to the second rack, and another end portion extending from the second rotating center of the second lever member and hinge-coupled to the second connection member.

20. The trocar holder of claim 18, wherein
the first rack and the second rack are arranged to intersect with the power transfer axis of the one shaft, and
upon rotation of the one shaft in one direction, the first rack moves in a longitudinal direction of the first rack, and the second rack moves in a longitudinal direction of the second rack, with one end portion of the first rack and one end portion of the second rack moving closer to or away from each other.
